(19) United States
(12) Patent Application Publication
(10) Pub. No.: US 2020/0312462 A1
(43) Pub. Date: Oct. 1, 2020

(54) METHOD FOR SIMULATING RESPIRATORY DYNAMICS OF A VIRTUAL LUNG WITH MODELLING OF THE MUSCULAR PRESSURE, VIRTUAL SIMULATOR

(71) Applicants: UNIVERSITE DE BORDEAUX, Bordeaux (FR); CENTRE HOSPITALIER UNIVERSITAIRE DE BORDEAUX, Talence Cedex (FR); FONDATION BORDEAUX UNIVERSITE, Bordeaux (FR)

(72) Inventors: Rémi DUBOIS, Mérignac (FR); Hadrien ROZE, Bordeaux (FR)

(21) Appl. No.: 16/649,490

(22) PCT Filed: Sep. 21, 2018
(86) PCT No.: PCT/EP2018/075594

§ 371 Date: Mar. 20, 2020

(30) Foreign Application Priority Data
Sep. 22, 2017 (FR) 1771004

(51) Int. Cl. G16H 50/50; G16H 50/20; G16H 50/30; G16H 40/67; G16H 40/40; G06F 30/28; A61M 16/00

(52) U.S. Cl. CPC G16H 50/50; G16H 50/20; G16H 50/30; G06F 2111/10; G16H 40/40; G06F 30/28; A61M 16/026; G16H 40/67

(57) ABSTRACT

A method for simulating respiratory dynamics of a virtual lung on the basis of a virtual lung model configured as a function of a first parameterization and at least one ventilation mode, includes a first configuration of the virtual lung model; a second configuration of a model of a virtual respiratory system, including a functional relationship between: a flow rate of air inhaled or exhaled by a virtual patient and; at least one considered pressure in the respiratory circuit, the considered pressure being a resulting pressure corresponding to the pressure in the respiratory tracts of the virtual lung, called output pressure, from which is subtracted an inner pressure in the virtual lung, the inner pressure of the virtual lung including a muscular pressure and the pressure inside the lung; a third configuration of at least one ventilation mode. The method includes generation of at least one curve.

### FIELD

The field of the invention relates to simulation methods and systems making it possible to train medical and paramedical personnel notably for the purposes of configuring respiratory assistance devices for given patients. The field of the invention notably relates to the modelling of the respiratory system, a virtual lung and predefined ventilation modes. The field of the invention pertains to simulators notably making it possible to generate pressure and volume plots of the lung which are faithful and representative of known pathologies.

### PRIOR ART

In the field of methods and devices for helping or assisting the respiration of a patient suffering from a given respiratory pathology, there is an increasing need to perform simulations in order to avoid the risks that inappropriate adjustments of equipment in real situations comprise.

A fortiori, equipment for which the operating modes or parameterizations constitute a risk during their handling by medical personnel require that learning the different operating modes thereof is carried out upstream. In order to learn how to operate a respirator and to take control of the different ventilation modes, tests and simulations may be carried out with patients in order to illustrate different operating points and the effects of respiratory treatments as a function of the pathologies to address.

One of the functions to simulate is the artificial respiration of acute respiratory distress syndrome. This corresponds to the use of a ventilatory assistance machine which replaces the function of the patient who is deficient for the time that the causal treatment takes effect.

However, this situation presents a discomfort, or even a danger, for the patients involved.

It is thus necessary to learn to adjust these machines for each pathology and to adapt the adjustments to the evolutive situation of patients. This corresponds to combinations of extremely numerous adjustments and an incorrect adjustment may directly affect mortality. The effects of adjustments of the ventilator by the physician on the mortality of patients with acute respiratory distress syndrome have been demonstrated for:
motor pressure
adjustment of the current volume
adjustment of the positive expiratory pressure
transpulmonary pressure.

In the operating room with anaesthetised patients without respiratory dysfunction, optimisation of the adjustments of the positive expiratory pressure and the current volume decreases postoperative respiratory morbidity.

In order to limit the dangers for patients, lung simulators exist which can interface with existing respirators in order that medical personnel can train themselves or learn to adjust the different operating modes.

However, these simulators comprise major drawbacks:
The lung simulation modes are simplified and do not make it possible to obtain a lung model faithful to the working of a real lung.
Moreover, the different simulation modes do not take into account a modelling of complex pathologies or particular physiological working of the lung, for example such as alveolar recruitment.
The artificial lung is not compatible with all the ventilation modes and must be the subject of particular adaptations on a case by case basis.
When a patient is assisted by a respirator, he or she may decrease the muscular effort to breathe.

Finally, lung simulators, usually mechanical, are costly and in general only take into account a limited number of patient profiles and ventilator adjustments.

Methods for modelling respiratory mechanics are known from the prior art. The solution described in the document WO9951292 illustrates a simulator of respiratory functions of a lung and the taking into account of ventilation mode.

However, this solution has the drawback of a linear approximation and a simplified model not making it possible to simulate the working of a lung over a wide range of values.

Finally, this solution does not make it possible to reproduce faithfully a major characteristic of the pulmonary system called ‘recruitment’ linked to the modification of the alveolar volume in certain patients assisted by a respirator. Moreover, the modelling of muscle pressure does not take into account the respiratory assistance that influences the value of muscle pressure.

### SUMMARY OF THE INVENTION

The method of the invention makes it possible to resolve the aforesaid problems.

The objective of the method of the invention is to develop a simulation of the thoracic function of a patient thus making it possible to reproduce the pathologies and critical situations which are the critical reality at the bed of the patient. The method could notably be implemented in a simulator comprising an interactive control screen, for example similar to those of commercially available ventilators, in order that the operator tests the adjustments of the ventilator and observes the consequences of his choices.

The interface will have the objective of offering an interaction to health professionals in the field of artificial ventilation. The medical teaching of respiratory pathologies could to a large extent become totally virtual and no longer necessitate the mobilisation of costly items of equipment, limited in their working and interactions.

According to a first aspect, the invention relates to a method for simulating respiratory dynamics of a virtual lung on the basis of a virtual lung model and at least one ventilation mode, characterised in that the method comprises:
a first configuration of the virtual lung model, said model comprising:
   A non-linear functional relationship between an instantaneous volume of the virtual lung and an instantaneous pressure of the virtual lung;
   A first parameterization of the non-linear function;
said first configuration comprising the determination of the following parameters:
   a maximum dynamic corresponding to a given air admission capacity of the virtual lung;
a second configuration of a model of a virtual respiratory system comprising a functional relationship between:
   on the one hand, a flow rate of air inhaled or exhaled by a virtual patient and;
   on the other hand, at least one considered pressure in the respiratory circuit, said considered pressure being a resulting pressure corresponding to the pressure in the respiratory tracts of the virtual lung, called output pressure, from which is subtracted an inner pressure in the virtual lung, the inner pressure of the virtual lung comprising a muscular pressure and the pressure inside the lung;
   the muscular pressure being determined as a function of a respiratory adaptation model comprising an adaptation coefficient weighting the value of said muscular pressure and determined as a function of the evolution of a value of a target parameter to reach;
said second configuration comprising the determination of a datum characteristic of at least one ventilatory resistance of a virtual patient;
a third configuration of at least one ventilation mode comprising the determination of at least one virtual respiratory cycle comprising at least an expiration phase and an inspiration phase, which phases being associated with a condition of evolution either of the flow rate of air exhaled and/or inhaled, or the output pressure,
the method comprises the generation of at least one curve representing a plot of the pressure within the virtual lung as a function of the volume of the virtual lung from the parameterized virtual lung model, the parameterized model of the respiratory system and a predefined ventilation mode.

According to an embodiment, the first configuration comprises, moreover, determination of:
an inflection pressure corresponding to the inflection point of the non-linear function and;
the slope of the non-linear function at the inflection point;
a corrective factor of an admission volume, called the recruitment factor.

According to another aspect, the invention relates to a method for simulating respiratory dynamics of a virtual lung on the basis of a virtual lung model and at least one ventilation mode, characterised in that the method comprises:
A first configuration of the virtual lung model, said model comprising:
   A non-linear functional relationship between an instantaneous volume of the virtual lung and an instantaneous pressure of the virtual lung;
   A first parameterization of the non-linear function;
said first configuration comprising the determination of the following parameters:
   a maximum dynamic corresponding to a given air admission capacity of the virtual lung;
a second configuration of a model of a virtual respiratory system comprising a functional relationship between:
   on the one hand, a flow rate of air inhaled or expired by a virtual patient and;
   on the other hand, at least one considered pressure (P) in the respiratory circuit;
said second configuration comprising the determination of a datum characteristic of at least one ventilatory resistance of a virtual patient;
a third configuration of at least one ventilation mode comprising the determination of at least one virtual respiratory cycle comprising at least an expiration phase and an inspiration phase, which phases being associated with a condition of evolution either of the flow rate of air exhaled and/or inhaled (Q), or of the output pressure, characterised in that:
the first configuration comprises, moreover, a determination of:
   a corrective factor of an admission volume, called the recruitment factor,
the method comprises generation of at least one curve representing a plot of the pressure within the virtual lung as a function of the volume of the virtual lung from the parameterized virtual lung model, the parameterized model of the respiratory system and a predefined ventilation mode.

The following embodiments relate to one or the other of the aspects of the invention.

According to an embodiment, the application of said recruitment factor generates an increase or a decrease in the slope at the inflection point of the functional relationship between the volume and the pressure of the virtual lung model.

According to an embodiment, the first configuration comprises,
moreover, a determination of:
   an inflection pressure corresponding to the inflection point of the non-linear function and;
   the slope of the non-linear function at the inflection point;

According to an embodiment, the functional relationship between the instantaneous volume of the virtual lung and the instantaneous pressure of the virtual lung is of sigmoid type.

According to an embodiment, the recruitment factor is determined as a function of a recruitment model comprising a parameterizable recruitment coefficient and dependent on a predefined value of a virtual base pressure introduced at the input of the virtual lung.

According to an embodiment, the recruitment factor comprises:
a first term which is a function of the virtual base pressure introduced into the virtual lung;
a second term which is a function of the virtual air pressure in the virtual lung and the virtual base pressure.

According to an embodiment, the recruitment factor is expressed by a linear relationship with the pressure in the lung:

K=C₁+C₂·P_{P},

Where, C₁ and C₂ are functions of the base pressure P_{PEP}.

According to an embodiment, the model of the respiratory system comprises:
the considered pressure of the respiratory circuit is a resulting pressure corresponding to the pressure in the respiratory tracts at the output of the virtual lung, called output pressure, from which is subtracted an inner pressure in the virtual lung, the inner pressure of the virtual lung comprising a muscular pressure and the pressure inside the lung;
the functional relationship between the flow rate of air inhaled or exhaled and the resulting pressure is linear, the linearity coefficient corresponding to the datum characteristic of the ventilatory resistance of the virtual patient.

According to an embodiment, the muscular pressure is determined as a function of a respiratory adaptation model comprising an adaptation coefficient weighting the value of said muscular pressure and determined as a function of the evolution of a value of a target parameter to reach.

According to an embodiment, the target parameter to reach is a target volume of the virtual lung to reach.

According to an embodiment, the ventilation mode is a first mode comprising an inspiration phase of the respiratory cycle configured with a constant air flow rate.

According to an embodiment, the ventilation mode is a second mode comprising an inspiration phase of the respiratory cycle configured with a constant output pressure.

According to an embodiment, the ventilation mode is a third mode comprising an inspiration phase of the respiratory cycle configured with a constant output pressure and of which the phase is engaged subsequent to the detection of an output pressure threshold exceeding a predefined pressure threshold.

According to an embodiment, the ventilation mode is a fourth mode comprising an inspiration phase of the respiratory cycle configured with an output pressure proportional to a setpoint, said setpoint being determined by the measurement of a physiological parameter, said physiological parameter being:
a pressure of the patient, or;
an electrical signal representative of a respiratory muscular effort.

According to an embodiment, a step of temporal discretisation of the virtual lung model and the model of the respiratory system is calculated for at least one given ventilation mode by considering at least one first respiratory phase wherein the flow rate of air exhaled and/or inhaled is constant and/or a second respiratory phase wherein the output pressure of the respiratory system is constant.

According to an embodiment, in the course of a respiratory phase during which the output pressure P_{AW} is considered as constant, the discretisation step comprises an approximation of a value maintained constant of the muscular pressure between two samples of the discretisation.

According to another aspect, the invention relates to a computer programme product comprising instructions which, when the programme is executed by a calculator, lead said calculator to implement the method of the invention.

According to another aspect, the invention relates to a computer readable recording support comprising instructions which, when they are executed by a calculator, lead said calculator to implement the method of the invention.

According to another aspect, the invention relates to a virtual simulator comprising at least one interface to configure the first, the second and the third configurations of the simulation method, said at least one interface being defined in a same portable equipment. The portable equipment may be a digital tablet or a smartphone.

According to another example, the equipment is a computer.

According to another aspect, the invention relates to a respiratory assembly comprising:
an intermediate ventilation device (DISPO_INT VENT) intended to engage mechanically with a ventilation system of a respirator (RESP) intended to assist a patient;
a virtual lung (VIRT_{P}) according to the invention generating numerical setpoints corresponding to an output pressure (P_{AW}) of a virtual respiratory system and an outgoing airflow rate (Q) according to a predefined respiratory cycle, the virtual lung (VIRT_{P}) and the respiratory cycle being configured according to the method of the invention, said numerical setpoints controlling the intermediate ventilation device.

### BRIEF DESCRIPTION OF THE FIGURES

Other characteristics and advantages of the invention will become clear from reading the detailed description that follows, with reference to the appended figures, which illustrate:

FIG. 1: a block diagram of the main models and interfaces enabling the implementation of an embodiment of the method of the invention;

FIG. 2: a plot of a curve generated according to an embodiment of the method of the invention linking the pressure of the lung and the volume of said lung;

FIG. 3: an example of interface of a simulator of the invention.

FIG. 4: an example of model of a virtual respiratory system designed by analogy with Ohm's law;

FIG. 5: an example of virtual lung interfacing with an intermediate device configured to engage mechanically with a respirator.

### DESCRIPTION

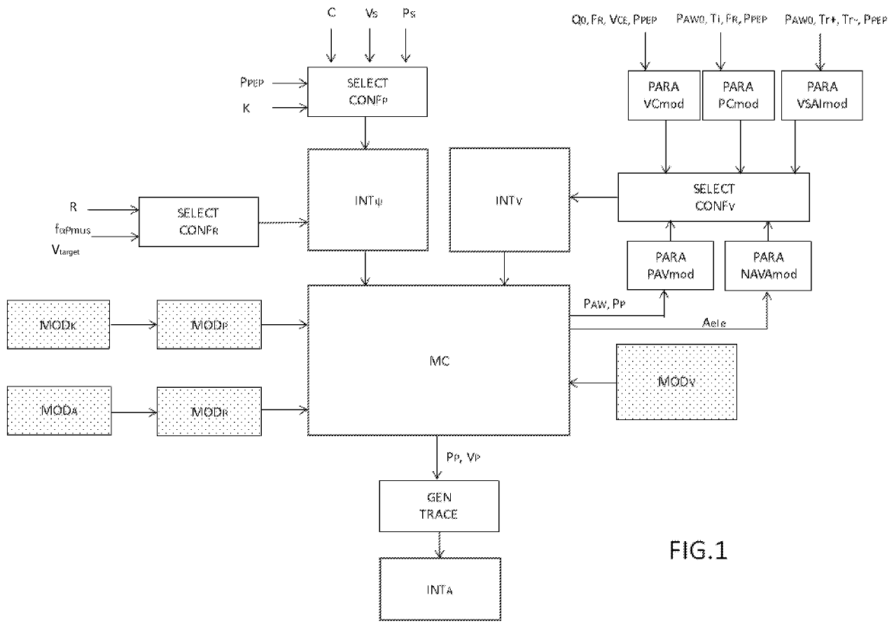

FIG. 1 illustrates the different elements making it possible to implement an embodiment of the invention.

Different data models may be used for the purposes of modelling the behaviours and evolutions of a virtual lung, a respiratory system and a virtual ventilator. Each model may be configured independently of each other. An objective is to provide a faithful modelling of a real respiratory assistance of a patient. This modelling offers a training tool for personnel capable of establishing connections between evolution curves of the respiration of virtual patients and pathologies. Moreover, the invention makes it possible to become familiar with equipment dedicated to the respiratory assistance of a patient and the effects of different ventilation modes.

Lung Model

A lung model MOD_{P} makes it possible to define a virtual lung, that is to say the working of a lung while taking into account different data making it possible to configure the model. It may be patient data relative to his or her age, corpulence, sex, etc. and/or pathology data relative to a maximum exhaled volume, a residual volume in the lung, a pulmonary capacity, a compliance (elasticity of the lung), etc.

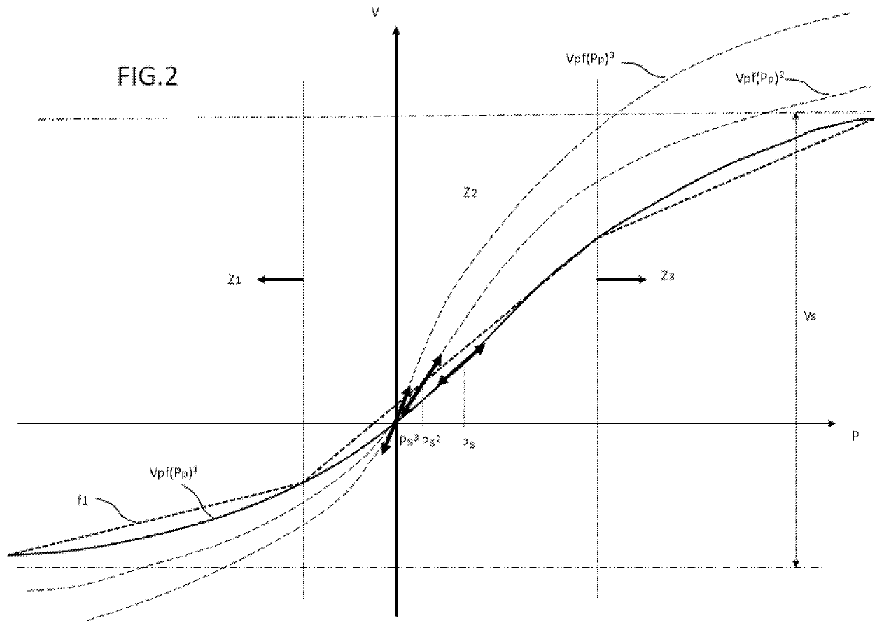

The lung model is noted MOD_{P} in FIG. 1. It is notably mainly characterised by a curve noted Vₚf(Pₚ) which is a non-linear function defining the evolution of the volume of the lung Vₚ as a function of the pressure Pₚ within said lung. According to an embodiment, this curve is non-linear. According to a first alternative, it may be of sigmoid type such as represented in FIG. 2. According to another alternative, it may be approached by a polynomial function (not represented). An advantage of the method of the invention is to take into account a relationship between the pressure and the volume V=f(P) faithful to the working of a real lung. The method makes it possible to take into account a non-linear curve due to the resolution by a discretisation of the problem to resolve described hereafter.

In the embodiment of a sigmoid type curve, this curve is obtained from the following equation [1]:

Vₚf(P_{P})=K·Vₛ/(1+e^{-Cs(P-Ps)})−A [1]

### Where:

P_{P} is the pressure in the lung at the instant t, also noted P(t);
K is a factor which is deduced from a recruitment model, called the recruitment factor. It may be considered as a corrective factor of an admission volume.
Cₛ represents the value of the slope of the sigmoid curve at the inflection point Pₛ. It may for example be expressed as a function of Vₛ.
Vₛ represents the value of the maximum dynamic considered for a given lung, that is to say an admissible volume of air calculated between a rest point and an attainable volume value of the lung during the inspiration phase.
“A” represents a given constant calculated as a function of at least one datum linked to the patient.

According to an embodiment, A is determined as a function of the values of the parameters of Pₛ, Cₛ and of Vₛ. According to an embodiment, the constant “A” may be a function of the coefficient k, for example in a linear relationship, a second degree or third degree type relationship.

An advantage of such a model is to represent a relationship linking the volume and the pressure of the lung which is faithful to the behaviour of a real lung. The sigmoid type curve makes it possible to obtain good faithfulness. A drawback resolved by the virtual lung model of the invention is that of imprecision which could generate a linear model f₁ of which an example of a piecewise linear curve in different pressure zones is also represented in FIG. 2.

According to an embodiment, parameters defining the profile and the plot of the non-linear function may simply be defined by an operator from an interface. The coordinates of the inflection point at the point Ps, the value of the maximum dynamic and the slope at the inflection point of the linear curve suffice to characterise a plot of the non-linear function, notably of sigmoid type. This possibility offers an advantage during the definition of the lung model MOD_{P}.

In FIG. 2, the x-axis represents the pressure, whereas the y-axis represents the volume.

The linear curve f1 is represented piecewise in three zones Z₁, Z₂ and Z₃.

The sigmoid type curve Vₚf(P_{P})¹ is also represented in the three zones.

The external zones Z₁ and Z₃ show differences between a sigmoid type curve Vₚf(P_{P})¹, also noted Vₚf(P_{P})1 in FIG. 1, and a linear curve f₁. Yet, it is known that the relationship linking the pressure P to the volume V of a lung forms an “S” of which a better approximation may be a sigmoid. A problem is that this function is difficult to resolve for a dynamic model. Hence, linear approximations enable a reasonable approximation. However it appears that, with such linear modelling, it becomes particularly difficult to take into account a wide variety of patient profiles in the model, moreover a linear model would be less accurate in the faithful reproduction of the working of a lung. The virtual lung model modelling a sigmoid type mathematical function is thus more faithful to the working of a real normal or pathologic lung from a modelling of the invention. It thus makes it possible to generate better simulation of the working of a lung.

The central zone Z₂ also shows a difference between the sigmoid type curve Vₚf(P_{P})¹ and the linear curve f₁. Furthermore, the curve of the invention Vₚf(P_{P})¹ makes it possible to define precisely the position of an inflection point Ps, the point Ps being a point characteristic of the sigmoid type curve.

An advantage is to be able to take into account an appreciation of a residual volume in the lung at the neutral point of the respiration in proportion with a total volume. The neutral point is the rest point at the end of expiration, that is to say when there is a base pressure imposed by the respirator, the point PEP(P_{PEP}, V_{PEP}). Here different types of patients may be modelled by considering that at the respiration neutral point an over-volume or an over-pressure of air is present in the lung.

The method of the invention makes it possible to take into account different parameterizations of a curve Vf(P), notably curves where the pressure Ps at the inflection point is different from 0. The value of the pressure Ps notably makes it possible to configure for a given patient a type of lung model. By default, this value may be determined in a configuration file on the basis of a set of predefined values and corresponding to predefined profiles.

For a linear approximation in the central zone Z₂, it is not possible to define the exact position of the inflection point Ps and this thus results in an impossibility of configuring a model of a virtual lung taking into account this datum.

An advantage of the definition of an inflection point Ps of the sigmoid is to be able to parameterize in the virtual lung model MOD_{P} a datum relative to a respiration neutral point according to a patient typology or pathology.

Recruitment Model

According to an embodiment of the invention, the method of the invention comprises a possible parameterization of an alveolar recruitment datum K in order to define an enriched virtual lung model MOD_{P} and which is representative of certain respiratory pathologies responsible for alveolar collapse.

The recruitment datum may be determined by a recruitment model MOD_{R} representative of the physiological phenomenon that it induces. Recruitment consists in reopening collapsed pulmonary territories with the aim of making ventilation more homogeneous. This recruitment is obtained by the set of adjustments that make it possible to increase the pressure in the respiratory system.

This modelling may be activated or not from a control interface of the simulator when the “patient” parameters are defined.

Recruitment leads to an increase or a decrease in the slope of the curve Vf(P), said slope being called the “compliance” of the lung. The recruitment may be modelled by a factor K, the expression of which is defined by the following relationship:

k=C₁+C₂·P [2]

This relationship may involve the pressure calculated at different places. Thus, P may be the pulmonary pressure P_{P}, the output pressure of the respiratory system P_{AW} or the base pressure P_{PEP}.

According to an embodiment, C₁ and/or C₂ are determined as a function of the base pressure P_{PEP}.

where:
C₁ and C₂ represent recruitment coefficients which can be normalized as a function of predefined values. For example, they may be normalized for a normal patient, that is to say a healthy lung of an average individual, of average size and age.
P_{PEP} represents the minimum air pressure imposed by a virtual ventilator at the input of the respiratory system, that is to say at an input of the virtual lung. This pressure P_{PEP} may be parameterized in order to provide an aid to respiration for certain patients in order to ensure a minimum pressure in the respiratory cycle.

It aims to take into account the working of a real ventilator and also a physiological phenomenon linked to its application.

The recruitment phenomenon, which is a physiological phenomenon, may stem in part from the application of the pressure P_{PEP}. This contribution to the recruitment phenomenon may be modelled while taking into account the value of the pressure P_{PEP} when it is imposed.

K may also be written thus: K=k₁+k₂

A first recruitment factor k₁ designates the proportion of recruitment that is associated with the application of the pressure P_{PEP}.

According to an embodiment, the term k₁ may be a linear function of the pressure P_{PEP}. According to another embodiment, k1 is perhaps a polynomial function of the pressure P_{PEP}. It is the proportion in the recruitment coefficient K induced by the application of the pressure P_{PEP}.

A second recruitment term k₂ designates a proportion of recruitment that is associated with the admission of additional air into the lung due to the application of pressure in the lung, which leads to reopening of a part of the collapsed pulmonary territories on account of alveolar recruitment. This additional recruitment phenomenon is the consequence of a double phenomenon: the inspired air increases the pressure but also reopens certain alveola and increases the pulmonary capacity, which leads to a local decrease in pressure. This recruitment term corresponds to the change in volume induced by the respiration and thus by the over-pressure vis-à-vis the pressure P_{PEP}. According to an embodiment, the second term k₂ is a function of the virtual air pressure P_{P} or the air volume Vₚ in the virtual lung and the virtual base pressure P_{PEP} or its associated volume V_{PEP}.

The curves Vₚf(Pp)² and Vₚf(Pp)³ of FIG. 2 illustrate the consequences of taking into account recruitment factors: a change of slope and a translation of the curve with respect to the curve Vₚf(Pp)¹ which does not take into account the recruitment phenomenon. This is illustrated by the evolution of the position of the inflection points of each curve at the point Pₛ² and Pₛ³.

This involves a recruitment phenomenon induced by respiratory mechanics. This term is taken into account in a certain type of virtual lung which depends on the profile {patient; pathology} of which it is wished to simulate the working.

The term k₂ of the recruitment K is the part induced by the modification of the instantaneous capacity of the virtual lung. It may be modelled by a linear function of the pressure P and also take into account a coefficient involving the base pressure P_{PEP}, for example by a function that is also linear.

An advantage of the virtual lung model of the invention thus stems from the capacity to model the recruitment phenomenon and thus to extend the modelling of pathologies and patients.

The curves Vₚf(P)², Vₚf(P)³ represent sigmoids obtained for subjects with recruitment. The absolute value of the recruitment factor K is modified to be greater than 1. The virtual lung model MOD_{P} thus varies as a function of the recruitment model MOD_{K} applied as illustrated in FIG. 2.

The recruitment phenomenon may lead to an increase in the pulmonary capacity but also a decrease in the pulmonary capacity, for example when the recruitment factor K is less than zero.

Interface, Configuration of the Lung Model

According to an embodiment, the invention comprises a parameterization of the lung model. To this end, a specific interface INTψ may be designed in order to adjust the different values of the parameters of the lung model MOD_{P}.

This interface makes it possible to define parameters of the curve Vf(P) of which notably the slope at the inflection point C, the maximum capacity of the lung Vs, the pressure at the inflection point Ps. According to another embodiment, the parameters may be loaded in the simulator from a configuration file. The file may be transferred or directly entered in the simulator.

Normalized values by default may be predefined to correspond for example to a healthy subject, that is to say without declared pathology and/or a reference subject who is representative of a nominal of an average subject of the population.

The pressure P_{PEP} of a virtual respirator may be defined.

Finally, the recruitment model MOD_{K} may be completely defined, notably by the determination of two recruitment factors k₁ or k₂ or by the two factors of the linear relationship defined previously C₁ and C₂. The recruitment K is also a function of the pressure in the lung P_{P}, however the relationship could also be written with the pressure P calculated at another point of the system, for example P_{AW}.

The functions SELECT_CONF_{P} and SELECT_CONF_{R} make it possible to take into account the value of a parameter and to inject it into the model. These functions may be realised by means of predefined values, a dropdown menu or instead an input field.

The data may be input from a tactile interface, for example a digital tablet or a smartphone. The input data are used by a calculator MC in order to generate with the other parameterizations a curve Vₚf(P) or instead other plots making it possible to monitor the evolution of a parameter linked to respiration.

Model of the Respiratory System of the Lung

According to an embodiment of the invention, a virtual respiratory system model MOD_{R} may be defined notably by a parameterization of values defining certain numerical conditions of the model MOD_{R}.

The virtual respiratory system model is defined by the following relationship:

P_{AW}−Pₘᵤₛ=R·Q+P_{P} [3]

### Where:

P_{AW} is the output pressure of the respiratory system of a virtual patient, that is to say the pressure measured by a virtual respirator connected in theory to a lung. The pressure P_{AW} takes into account the pressure of the respiratory system and the pressure derived from physiological tubes up to the input of the respirator.
Pₘᵤₛ is the muscular pressure of the lung. It corresponds to the muscular pressure generated by a muscular effort of the lung of a patient to breathe.
Q is the flow rate of air expulsed or inhaled at the output of the respiratory system of a virtual patient.
P_{P} is the pressure in the lung.
R is a ventilatory resistance. It may correspond to the sum of the resistance of the patient and the resistance of the respirator. The resistance of the artificial ventilatory system of the respirator takes, for example, into account that of the tubes, the valves, and any respiration accessory present in the ventilation circuit.

The term inner pressure Pi is employed to designate the pressure of the lung P_{P} and the muscular pressure Pₘᵤₛ.

Thus, the flow rate Q multiplied by the respiratory resistance may be seen as a resultant between the output pressure P_{AW} and the inner pressure. R·Q=P_{AW}−Pᵢ.

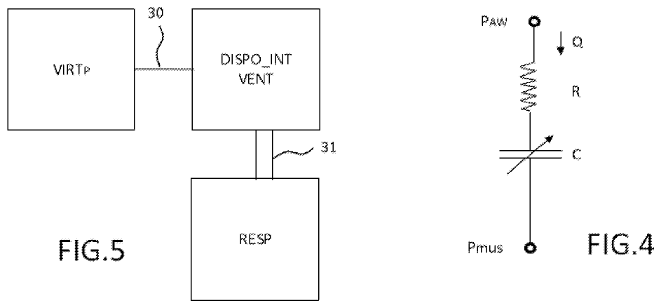

The model is established by analogy with Ohm's law. FIG. 4 represents a diagram of an electric circuit of which the calculation of the difference in potentials at the terminals makes it possible to deduce a relationship between the current I, the resistance R, a capacitance C and said difference in potentials. By analogy, the resistance R may be assimilated with the respiratory resistance R (same notation), the current I at the air flow rate Q, the capacitance at the instantaneous pulmonary pressure Pₚ(t) and the difference in potentials at the difference in pressures at the two external points of the respiratory system P_{AW}−Pₘᵤₛ at the difference in potentials of the circuit. An external point may be considered as the muscle of the lung and the other point as the output of the respiratory system.
The relationships linking the flow rate and the volume are also known, since the air flow rate Q is a derivative of the volume V with respect to time t:

Q=dV/dt [4]

And the relationship defined previously [1]:

Vₚf(P_{P})=K·Vₛ/(1−+e^{-Cs·(P-Ps)})+A [1]

may thus be expressed in different ways according to the considered pressure, P_{AW}, Pₘᵤₛ, or any other pressure which can be measured in the respiratory circuit due to the relationship [3] which links the pressure in the lung P_{P} to a pressure P_{AW} or Pₘᵤₛ. Generally speaking, the relationship Vₚf(P) could be evoked in the present description, to signify that the volume of the lung Vₚ may be a function of a pressure measured at a given point of the ventilation circuit.

More generally, a relationship Vf(P) will be employed to characterise a lung model.

An advantage of the respiratory model of the invention is to take into account the muscular pressure Pₘᵤₛ. The invention is also based on a modelling of the respiratory resistance of patient profiles according to their pathology or pathologies, their age, their sex, etc.

According to an embodiment, the respiratory system model of the invention takes into account dynamic modelling of the muscular pressure Pₘᵤₛ. This modelling may be activated or not from a control interface of the simulator. According to an embodiment, a configuration file may be prepared remotely or directly on the simulator to be exploited by the calculator of the simulator during the generation of the curves, notably Vₚ=f(Pₚ) and/or V_{AW}=f(P_{AW}).

Model of Muscular Pressure Pₘᵤₛ

An advantage of the invention is to take into account a faithful model of the muscular pressure of a patient who could be assisted by a respirator. One advantage is to take into account more precisely phenomena actually arising in real patients by reconstituting a pairing {virtual patient; virtual respirator} which can be configured according to a given parameterization.

When a patient is assisted by a respirator, he can reduce his muscular effort to breathe, notably in certain conditions relative to a given patient profile and/or a given pathology.

In order to take account of this phenomenon, a quantity may be adjusted to weight a value Pₘᵤₛ representative of certain cases.

According to an embodiment, the weighted value is a level of gas, which may be for example carbon dioxide assimilated per unit of volume. According to another example, the weighted value may be an oxygen level. The definition of this variable to adjust makes it possible to take into account the fact that the patient adjusts his muscular pressure Pₘᵤₛ to guarantee a given volume·minute⁻¹ of gas, noted V_{target}.

According to other embodiments, the muscular pressure Pₘᵤₛ may be defined according to another variable to adjust.

According to an embodiment, the expression of the weighted muscular pressure may be expressed thus:

Pₘᵤₛ(t)=<Pₘᵤₛ(t)>(1+α(t)) [5]

### Where:

<Pₘᵤₛ(t)> is a theoretical pressure curve.
α(t) is an adaptation coefficient which defines the muscular pressure model MOD_{A}, notably represented in FIG. 2.

When α(t)) is positive, the patient increases his muscular effort to reach the volume V_{target}, whereas when α(t)) is negative the patient reduces his muscular effort to reach the volume V_{target}. The parameter V_{target} is a virtual physiological setpoint which simulates a real phenomenon, notably the fact that a real patient enslaves his muscular effort to have a certain given volume·minute⁻¹. Thus, the muscular pressure model according to an aspect of the invention makes it possible to offer a rich model of the lung and the respiratory dynamics.

During the respiratory cycle or at the end of each respiratory cycle, the calculation of the volume of the lung may be expressed in Is⁻¹ and may be described thus:

V_{AW-LOCAL}=V_{AW}·fᵣₑₛₚ [6]

### Where:

fᵣₑₛₚ is the respiration frequency.

Such an expression makes it possible to calculate step by step, by discretising the relationship [6], the moment where one approaches the target volume V_{target}. The calculation of V_{AW-LOCAL} is done then is compared with the volume V_{target}. By discretising the relationship [6] between two different instants, one obtains:

α(n)=α(n−1)+1/f_{αPmus}·(V_{ntarget}−V_{nlocal})/V_{ntarget} [7]

### Where:

f_{αPmus} is an adaptation factor, said factor makes it possible to take into account in the modelling the rate of convergence towards the target volume V_{target}. Thus, the greater the value of the adaptation factor f_{αPmus} the slower the convergence towards V_{target}.
   When V_{ntarget}=V_{nlocal}, one indeed has α(n)=α(n−1)

An advantage of taking into account the muscular pressure model Pₘᵤₛ is to simulate cases, thanks to the simulation method of the invention, linked to certain pathologies or certain patient profiles wherein the respirator induces a modification of the muscular pressure. Furthermore, certain spontaneous ventilation modes estimate Pₘᵤₛ to deliver respiratory assistance. The method of the invention as well as the simulator thus make it possible to take account of a wide variety of modes thanks notably to the modelling of Pₘᵤₛ.

Thus, such a respirator can illustrate numerous different patient profiles and situations while offering the most faithful possible simulation with respect to real cases.

The data of the model of muscular pressure f_{αPmus} and V_{target}, as well as the respiratory resistance R may be input from a tactile interface, for example a digital tablet or a smartphone. According to another example, the data may be input in a configuration file in order to be recorded in a memory of the simulator. The input data are used by a calculator MC in order to generate, with the other parameterizations, a curve Vₚf(Pₚ) taking into account the respiratory model of equation [3].

Virtual Respirator

According to an embodiment, the method of the invention comprises the modelling of a virtual respirator and thus different ventilation modes being able to be configured.

The virtual respirator may be assimilated with a choice of a given ventilation mode.

VC_{mode}

According to an embodiment, a first ventilation mode VC_{mode} may be configured. This mode is a volume mode controlled by a virtual respirator making it possible to reproduce the working of a real respirator wherein the control may be carried out by adjusting the input volume.

This mode comprises different phases each corresponding to a given moment of the respiratory cycle.

A first phase of insufflation is modelled by defining an insufflation at constant flow rate wherein Q=Q₀. This phase is managed by a volume to reach V_{CE} which may be configured during the parameterization of the mode VC_{mode}.

A second phase, optional, of inspiratory pause is modelled by defining an insufflation at constant flow rate wherein Q=0. This phase makes it possible to measure end inspiratory plateau pressure values P_{Plateau}. This phase makes it possible to evaluate the alveolar pressure of the respiratory system. The duration of this phase may be defined for example during the parameterization of the mode VC_{mode}.

A third free expiration phase is modelled by the determination of an output pressure P_{AW} of the respiratory system chosen as constant. When the output pressure of the respiratory system is imposed by the virtual respirator, it is equal to the pressure P_{PEP}.

A fourth phase, optional, of respiratory pause corresponding to the end of the expiration phase is modelled by a constant zero flow rate, Q=0. The duration of this fourth phase may be defined in the configuration of the mode VC_{mode}.

The second and fourth phases are optional and may be implemented or not by the simulation method of the invention. An advantage of these optional steps is to make it possible to carry out measurements in real time. Moreover, these phases make it possible to break down the respiratory cycle in a clear manner in order to visualise simulated phenomena for example for training purposes.

The second phase and the fourth phase may be configured such that the value of the setpoint relative to the respiratory frequency FR is fixed.

According to an embodiment, the configuration of the VC_{mode} comprises the definition of the following parameters:
Fr: respiratory frequency, expressed in cycles/min;
Q: flow rate, expressed in litres per minute;
VCE: volume to inhale, in ml
P_{PEP}: value of the base pressure P_{PEP} imposed by the virtual respirator.

According to an alternative embodiment, the mode VC_{mode} may be configured with a trigger initiating at a given pressure threshold or a given flow rate threshold, for example, if Pₘᵤₛ is different from zero.

The condition to trigger may be for example expressed thus:
   |ΔP_{AW}|>threshold₁, or;
   |ΔQ|>threshold₂.

An advantage of this mode is to simulate a minimum respiratory assistance paced by the respiratory cycle, whatever the differences in muscular efforts of the patient.

PC_{mode}

According to an embodiment, a second ventilation mode PC_{mode} may be configured. This mode is a pressure mode controlled by a virtual respirator making it possible to reproduce the operation of a real respirator wherein the control may be carried out by adjusting the pressure at the input.

This mode comprises different phases each corresponding to a given moment of the respiratory cycle.

A first phase of insufflation is modelled by defining an insufflation at constant pressure wherein P_{AW}=P_{AW0}. This phase is managed by a volume to reach V_{CE} which may be configured during the parameterization of the mode VC_{mode}.

A second phase of inspiratory pause is modelled by defining an insufflation at constant flow rate wherein Q=0. This phase makes it possible to measure end inspiratory plateau pressure values P_{Plateau}. This phase makes it possible to evaluate the alveolar pressure of the respiratory system. The duration of this phase may be defined for example during the parameterization of the mode PC_{mode}.

A third free expiration phase is modelled by the determination of an output pressure of the respiratory system P_{AW} chosen as constant when it is applied. The respiration of the patient is left free. When the output pressure P_{AW} of the respiratory system is imposed by the virtual respirator, it is equal to the pressure P_{PEP}.

A fourth phase of respiratory pause corresponding to the end of the expiration phase is modelled by a zero constant flow rate, Q=0. The duration of this fourth phase may be defined in the configuration of the mode VC_{mode}.

The second and the fourth phases are optional and may be implemented or not by the simulation method of the invention. An advantage of these optional steps is to make it possible to carry out measurements in real time. Moreover, these phases make it possible to break down the respiratory cycle in a clear manner in order to visualise simulated phenomena, for example for training purposes.

According to an embodiment, the configuration of the PC_{mode} comprises the definition of the following parameters:
Ti: inspiration time, expressed in seconds;
Fr: respiratory frequency, expressed in cycles/min;
Pc+: pressure to impose by the virtual respirator in addition to the base pressure P_{PEP};
P_{PEP}: value of the base pressure P_{PEP} imposed by the virtual respirator.

According to an alternative embodiment, the mode PC_{mode} may be configured with a trigger initiating at a given pressure threshold or a given flow rate threshold, for example, if Pmus is different from zero.

The condition to trigger may for example be expressed thus:
   |ΔP_{AW}|>threshold₁, or;
   |ΔQ|>threshold₂.

An advantage of this mode is to simulate a minimum respiratory assistance paced by the respiratory cycle whatever the muscular efforts of the patient.
VSAI_{mode}

According to an embodiment, a third ventilation mode VSAI_{mode} may be configured. This mode is a mode controlled by the detection of a spontaneous ventilation in the patient. To this end, an event trigger, also simply called trigger, is configured to set in motion an operating mode of the virtual respirator according to a given step of the respiratory cycle.

This mode is particularly interesting when it is used with a patient making a spontaneous respiration effort, for example when he is not apnoea anaesthetized or when he is able to make a respiratory effort.

After the detection of a volume of air inhaled per minute, that is to say a given flow rate Q₁, a first Trigger Tr+ is generated, a theoretical volume of air is then expelled by the virtual respirator which is configured to stop a constant pressure Pc+ in the phase corresponding to insufflation. The constant pressure is parameterized, it is noted Pc+ and it corresponds to the insufflation pressure in addition to the pressure P_{PEP}.

In this case, the virtual respirator pressure controls the exchanged air.

This mode comprises different phases, each corresponding to a given moment of the respiratory cycle.

A first insufflation phase is modelled by defining an insufflation at constant pressure wherein P_{AW}=P_{AW0}. This phase is initiated after the Trigger initiated.

A second trigger Tr− is configured to detect the end of the first phase. This second trigger may be defined for a value derived from the maximum insufflation flow rate of each cycle. It is defined as a percentage of this maximum insufflation flow rate specific to each cycle.

A phase following the first phase, designated free expiration phase, is modelled by the determination of an output pressure of the respiratory system Pc+ chosen as constant. The third phase ends when the trigger Tr+ is initiated for a given volume of air per min, or a given flow rate

A fourth phase, optional, of respiratory pause corresponding to the end of the expiration phase is modelled by a zero constant flow rate, Q=0.

According to an embodiment, the configuration of the VSAI_{mode} comprises the definition of the following parameters:
Tr+: initiation trigger in [l/min];
Tr−: trigger of end of insufflation in [%];
Pc+: pressure insufflated in addition to the pressure P_{PEP};
P_{PEP}: value of the base pressure P_{PEP} imposed by the virtual respirator.

PAV_{mode}

The ventilation mode PAV_{mode} makes it possible to re-loop the setpoint of the respirator on a pressure measurement in the patient Pₚ or P_{AW}. An advantage is to simulate a ventilation mode wherein the respirator provides an aid to respiration according to a setpoint proportional to the estimated pressure of the patient.

The mode PAV_{mode} may be an improved mode of the mode VSAI_{mode} with an initiation trigger Tr+,

The interest is to establish a “proportional” respiratory aid totally configured on the respiratory dynamics of the patient (defined by the equation of the movement of the lung, equation [3]) which can change within a same respiration cycle.

The respiration cycles may be identical to those of the mode VSAI_{mode}, that is to say of which the inspiration phase is carried out with a pressure or flow rate setpoint and for example a free expiration.

A second phase of inspiratory pause with respect to the mode VSAI may be defined. This inspiratory pause phase is modelled by defining an insufflation at constant flow rate wherein Q=0. The duration of this phase may be defined for example during the parameterization of the mode PAV_{mode}. It may be suspended if an insufflation is detected by the virtual patient. This pause makes it possible to measure the compliance of the respiratory system as well as the total resistances in order to resolve the equation [3].

NAVA_{mode}

The ventilation mode NAVA_{mode} makes it possible to re-loop the setpoint of the respirator on an electrical measurement of a muscular effort representative of a respiratory effort of the patient. It may be an electrical activity Aₑₗₑ, as is illustrated in FIG. 1, of the muscle of the diaphragm. An electrode comprised for example on the surface of a medical device may be configured to measure an electrical signal on the surface of the diaphragm. The electrical signal, depending on the measured level, may lead to the initiation and the ending of a suitable setpoint of the respirator. The inspiratory aid is synchronised with the electrical activation signal of the diaphragm.

An advantage is to simulate a ventilation mode wherein the respirator provides an inspiratory aid according to a setpoint proportional to a muscular electrical activity measured in the patient which is representative of an effort of the patient to engage his respiration.

The mode NAVA_{mode} may be an improved mode of the mode VSAI_{mode} with an initiation trigger Tr+. The initiation of the respiration cycles is defined by a certain electricity value but may be identical to those of the mode VSAI_{mode}, that is to say of which the phase of initiation of inspiration is realised with a pressure or flow rate setpoint. Expiration is initiated when the electricity reaches a certain % of the maximum inspiratory electricity. It is followed by a free expiration.

The choice of a ventilation mode may be determined with a view to simulating an operating mode of a respirator of which the configuration is adapted to a given pathology, a given patient.

An interface INT_{V} may make it possible to define the different modes and the associated parameters. This interface may be tactile. According to an embodiment, it may be generated on a same display as the interface INTLψ. The functions making it possible to choose the ventilation mode and the different parameters are represented in FIG. 1:
The function SELECT_CONF_{V} makes it possible to define the ventilation mode;
The functions PARA_VC_{mod}, PARA_PC_{mod}, PARA_VSAI_{mod}, PARA_PAV_{mod}, PARA_NAV_{Amod} make it possible to define the parameters of each respiratory phase for each of the ventilation modes.

These functions may be ensured by menus comprising predefined values or input fields.

Resolution of the System of Equations

An advantage of the invention is that the method takes into account different models and certain hypotheses in order to generate a system of equations which can be resolved in real time when the simulation is launched.

In order to resolve the system, the equation [1] is expressed according to the different possibilities of configuration of the virtual respirator and thus respiratory phases corresponding to the different ventilation modes.

For Respiratory Phase at Flow Rate Constant, Q=Q₀

It is possible to establish the following system of equations:

Q=dVₚ/dt [4]

P_{AW}−Pₘᵤₛ=R·Q+Pₚ [3]

Vₚf(Pₚ)=k·Vₛ/(1+e^{-Cs·(P-Ps)})+·A [1]

By discretising, at t=n and ΔT being the duration between and n and (n−1), one obtains:

Q(n)=Q₀

Vₚ(n)=Vₚ(n−1)+Q₀·ΔT

V_{AW}(n)=V_{AW}(n−1)+Q₀·ΔT

P_{AW}(n)=R·Q₀+P_{P}f(Vₚ(n))+Pₘᵤₛ(n)

Pₚ(n)=P_{P}f(Vₚ(n))

For Respiratory Phase at Constant Pressure, P_{AW}(t)=P_{AW0}

P_{AW}(t)−Pₘᵤₛ(t)=R·Q+P_{P}f(Vₚ)=R·dVₚ/dt+P_{P}f(Vₚ)

In order to resolve this equation, the method of the invention may comprise the formation of a first hypothesis of the model defining an approximation when one is in the proximity of Vₚ(n):

P_{P}f(Vₚ)=1/C_{LOC}·Vₚ,with C_{LOC}= [symbol] Vₚf/ [symbol] P_{P}(P_{P}),

Where [symbol] Vₚf/ [symbol] P_{P} is the partial derivative of Vₚf with respect to the pressure P_{P}.

In this way a first order linear equation is obtained, which is the simplest to resolve.

The method of the invention may comprise the determination of a second hypothesis in the elaboration of the model in order to obtain an efficient and faithful system modelling the evolution of muscular pressure locally. The second hypothesis gives a condition on the value of the muscular pressure Pₘᵤₛ in the vicinity of t=n·T. It is considered according to this second hypothesis that Pₘᵤₛ remains substantially constant between (n−1)T and nT.

An advantage of this approximation is to obtain an equation which may be easily resolved.

Indeed, by locally fixing, between two consecutive instants of discretisation, the value of Pₘᵤₛ the first order linear equation has a second constant term. Hence, the difference P_{AW}−Pₘᵤₛ is constant locally. The resolution is thereby thus simplified.

The values of PAW(n), Vp(n), Q(n), Pp(n) and VAW(n) are then obtained.

Simulator

Different Simulators

According to an embodiment, the simulator is configured to simulate the thoracic function of a patient and the function of the respirator.

According to another embodiment, the simulator VIRT_{P} is configured to simulate the thoracic function of a patient in order to test a real respirator RESP. In this case, as illustrated in FIG. 5, an intermediate ventilation device DISPO_INT_VENT may be used in order to be:
on the one hand, managed by the numerical setpoints 30 delivered by the simulator VIRT_{P} and:
on the other hand, mechanically interfaced with the ventilator of the respirator RESP.

The interfaces 31 with the respirator RESP then comprise physical channels to generate a flow of air or to inhale a flow of air according to the conditions imposed by the simulator.

According to an embodiment, the virtual simulator and the intermediate device are in the same equipment.

According to an embodiment, the virtual simulator only comprises the models and parameters necessary to simulate the thoracic and respiratory functions, that is to say the lung model MOD_{P}, the first configuration CONF_{P}, the respiratory system model MOD_{R} and the second configuration CONF_{R} as well as the parameters by default or the parameters to define from the interface.

According to an embodiment, the simulator of the invention makes it possible to model a virtual respirator, the functions making it possible to define the different ventilation modes. A pre-configuration may be generated in the simulator in order that it can be configured simply by the definition of a parameter in an input interface. According to an embodiment, the simulator comprises a memory making it possible to store the different models as well as the set of parameters by default.

According to an embodiment, the simulator comprises a single display INT_{A} to configure the first configuration CONF_{P} and the second configuration CONF_{R}. According to this same embodiment, the simulator comprises a single display making it possible to display the curves Vf(P) and the different control parameters being able to be controlled, whether they are those of the virtual lung or those of the virtual respirator.

Data Associated with the Simulator

An advantage of the simulator is that it may be associated with a local database or remote server comprising data representative of a set of pathologies or critical situations which are the clinical reality in the bed of the patients. The data may also comprise patient type profiles.

The database is defined so as to group together the parameters of configurations with given pathology contexts or given patient profiles. A set of parameters of the system of differential equations may be predefined for each of the pathologies. This solution makes it possible to predefine adjustments in order to generate plots corresponding to a patent context or pathologies.

Calculation Means of the Simulator

An interest of the simulator of the invention is to comprise a mathematical modelling of the movement of the lung (WP1) and the adjustment of the parameters of said model.

A first task of this WP consists in writing the differential equations that govern the thoracic system, then discretising them to use a numerical solver. A calculator such as a microprocessor may be used to perform the resolution and the discretisation of the latter.

The simulator comprises at least one calculation means MC making it possible to carry out the main calculation steps of the method. A specific or identical calculator for generating the curves may be used. The function GEN TRACE of FIG. 1 makes it possible to plot an evolution curve notably of V_{P} as a function P_{P} taking into consideration the different parameterized models. According to an embodiment, the parameterization or the modification of a model may be taken into account during the plot of the curve in a dynamic manner in order to be able to illustrate on a same graph the different curves changing as a function of the modifications made.

According to an embodiment, a programming of the evolutions of a parameterization may be carried out over a given period and over a range of predefined values of the parameter in order to vary the curve in real time with the modifications of the parameterization.

Such a programming is particularly didactic and pedagogical for illustrating the causal relationships between a patient or pathology modelling and a physiological response.

An advantage of the use of the method of the invention is to provide a simulator offering a realistic simulation of the output curves in relationship with a given pathology or a given patient profile.

According to an embodiment, physiological monitoring tools or adjustments are integrated in the simulator. The data and their interaction are calibrated from tests comprising comparisons with known clinical studies.

Display, Interface

According to an embodiment, the simulator comprises a display INT_{A} such as an interactive control screen. Advantageously, the interactive screen may comprise ergonomic elements similar to ventilators known to those skilled in the art.

Such a simulator enables an operator to test the adjustments of the ventilator and to observe the consequences of his choices.

The interface offers an interaction for health professionals in this field of artificial ventilation. Such a simulator makes it possible to dispense with the use of complex devices used.

Given the resolution of the system of equations, the method can generate all values representing a state of respiration: volume, pressure or flow rate of air at different places such as for example at the level of the muscle, the lung, at the output of the lung at the level of the respiratory system or instead at the level of the respirator. It is thus possible to access P_{AW}, V_{AW}, P_{P}, V_{P}, Pₘᵤₛ, V_{PEP}.

According to an embodiment, the simulator comprises selectors making it possible to choose and to configure a display mode of the different values representative of the respiration mode.

Notably, derivative values of these parameters expressed from a defined reference may be deduced from these values. Typically the pressure of the lung may be expressed with respect to the pressure of the muscle Pₘᵤₛ, with respect to atmospheric pressure Pₐₜₘ or instead with respect to any pressure.

Thus, a simple personalization of the display makes it possible to view the alveolar pressure, the trans-pulmonary pressure Pₘᵤₛ and for example the volume obtained by the base pressure P_{PEP} at the end of expiration. The display of these parameters makes it possible to provide a simulator offering numerous didactic display modes and a control tool illustrating:
all the implications of a modification of the ventilation mode in a given patient and;
all the implications of a passage from one patient profile to another profile in a given ventilation mode.

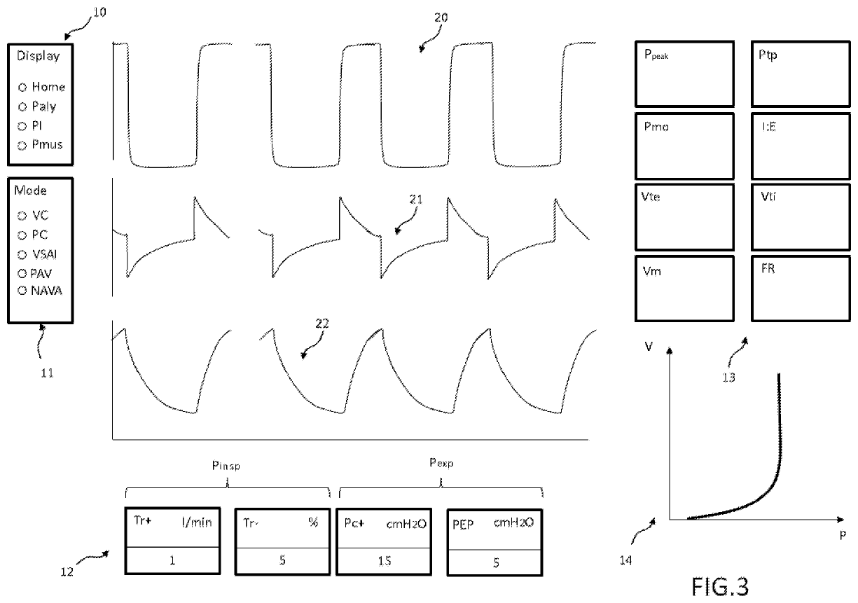

FIG. 3 represents an example of interface of the simulator of the invention comprising different zones making it possible to define a given parameterization. The zone 10 makes it possible to define parameters relative to the model of the respiratory system, of which the muscular pressure Pₘᵤₛ. The zone 11 makes it possible to define parameters relative to the model of the respirator and thus to the different ventilation modes of which the modes: VC_{mode}, PC_{mode}, VSAI_{mode}, PAV_{mode}, NAVA_{mode}. The zone 12 makes it possible to define parameters of the respirator such as the thresholds of the triggers Tr+, Tr−, the constant pressure Pc+ and the base pressure P_{PEP}. The zone 13 makes it possible to define data relative to the respiratory cycle such as the respiratory frequency FR, target volumes and parameters of conditions at the limits.

The zone 14 offers the plot of a reference curve making it possible to obtain a zone for controlling a curve of the volume of the lung V (P) as a function of the pressure of the lung P. Alternatively, an equivalent curve at the output of the respiratory system V_{aw}=f(P_{AW}) may be generated and displayed at the level of the zone 14. According to an embodiment, the two curves V_{P}=f(P_{P}) and V_{aw}=f(P_{AW}) may be displayed simultaneously.

The zones 20, 21, 22 enable the plot pf curves illustrating the evolution of certain parameters. The display may be configured so as to select or remove given plots.

One interest of the simulator of the invention is thus to propose integration of the model in computer software, with an interface screen for controlling the ventilator comprising arrangements and ergonomics facilitating handling by an operator or a physician.

The objective is to offer a unique pedagogical tool to health professionals or to manufacturers and which is simple to use.

1. A method for simulating respiratory dynamics of a virtual lung on the basis of a virtual lung model and at least one ventilation mode, the method comprising:
performing a first configuration of the virtual lung model, said model comprising:
a non-linear functional relationship between an instantaneous volume of the virtual lung and an instantaneous pressure of the virtual lung;
a first parameterization of the non-linear function;
said first configuration comprising the determination of the following parameters:
a maximum dynamic corresponding to a given air admission capacity of the virtual lung;
performing a second configuration of a model of a virtual respiratory system comprising a functional relationship between:
a flow rate of air inhaled or exhaled by a virtual patient and;
at least one considered pressure in the respiratory circuit, said considered pressure being a resulting pressure corresponding to the pressure in the respiratory tracts of the virtual lung, called output pressure, from which is subtracted an inner pressure in the virtual lung, the inner pressure of the virtual lung comprising a muscular pressure and the pressure inside the lung;
the muscular pressure being determined as a function of a respiratory adaptation model comprising an adaptation coefficient weighting the value of said muscular pressure and determined as a function of the evolution of a value of a target parameter to reach;
said second configuration comprising the determination of a datum characteristic of at least one ventilatory resistance of a virtual patient;
performing a third configuration of at least one ventilation mode comprising the determination of at least one virtual respiratory cycle comprising at least an expiration phase and an inspiration phase, which expiration and inspiration phases being associated with a condition of evolution either of the flow rate of air exhaled and/or inhaled, or of the output pressure,
the method comprising generating at least one curve representing a plot of the pressure within the virtual lung as a function of the volume of the virtual lung from the parameterized virtual lung model, the parameterized model of the respiratory system and a predefined ventilation mode.

2. The method according to claim 1, wherein the functional relationship between the flow rate of air inhaled or exhaled and the resulting pressure is linear, the linearity coefficient corresponding to the datum characteristic of the ventilatory resistance of the virtual patient.

3. The method according to claim 1, wherein the target parameter to reach is a target volume of the virtual lung to reach.

4. The method according to claim 1, wherein the first configuration further comprises the determination of:
an inflection pressure corresponding to the inflection point of the non-linear function and;
the slope of the non-linear function at the inflection point;
a corrective factor of an admission volume, called the recruitment factor.

5. The method according to claim 4, wherein the functional relationship between the instantaneous volume of the virtual lung and the instantaneous pressure of the virtual lung is of sigmoid type.

6. The method according to claim 4, wherein the recruitment factor is determined as a function of a recruitment model comprising at least one parameterizable recruitment coefficient and dependent on a predefined value of a virtual base pressure introduced at the input of the virtual lung.

7. The method according to claim 4, wherein the recruitment factor comprises:
a first term which is a function of the virtual base pressure introduced into the virtual lung;
a second term which is a function of the virtual air pressure in the virtual lung and the virtual base pressure.

8. The method according to claim 7, wherein the recruitment factor is expressed by a linear relationship with the pressure in the lung:
K=C₁+C₂·P_{P},
Where C₁ and C₂ are functions of the base pressure P_{PEP}.

9. The method according to claim 1, wherein the ventilation mode is a first mode comprising an inspiration phase of the respiratory cycle configured with a constant flow rate of air.

10. The method according to claim 1, wherein the ventilation mode is a second mode comprising an inspiration phase of the respiratory cycle configured with a constant output pressure.

11. The method according to claim 1, wherein the ventilation mode is a third mode comprising an inspiration phase of the respiratory cycle configured with a constant output pressure and of which the phase is engaged subsequent to the detection of an output pressure threshold exceeding a predefined pressure threshold.

12. The method according to claim 1, wherein the ventilation mode is a fourth mode comprising an inspiration phase of the respiratory cycle configured with an output pressure proportional to a setpoint, said setpoint being produced by the measurement of a physiological parameter, said physiological parameter being:
a pressure of the patient, or;
an electrical signal representative of a respiratory muscular effort.

13. The method according to claim 1, comprising calculating a step of temporal discretisation of the virtual lung model and the model of the respiratory system for at least one given ventilation mode by considering at least one first respiratory phase wherein the flow rate of air exhaled and/or inhaled is constant and/or a second respiratory phase wherein the output pressure of the respiratory system is constant.

14. The method according to claim 13, wherein in the course of a respiratory phase during which the output pressure is considered as constant, the discretisation step comprises an approximation of a value maintained constant of the muscular pressure between two samples of the discretisation.

15. A computer programme product comprising instructions which, when the programme is executed by a calculator, lead said calculator to implement the method according to claim 1.

16. A non-transitory computer readable recording medium comprising instructions which, when they are executed by a calculator, lead said calculator to implement the method according to claim 1.

17. A virtual simulator comprising at least one interface to configure the first, the second and the third configurations of the simulation method according to claim 1, wherein said at least one interface is defined in a same portable equipment.

18. A respiratory assembly comprising:
an intermediate ventilation device configured to engage mechanically with a ventilation system of a respirator configured to assist a patient;
a virtual lung, called virtual simulator, according to claim 17 generating numerical setpoints corresponding to an output pressure of a virtual respiratory system and an outgoing air flow rate according to a predefined respiratory cycle, the virtual lung and the respiratory cycle being configured according to the simulation method, said numerical setpoints controlling the intermediate ventilation device.
